(19) United States
(12) Plant Patent Application Publication
(10) Pub. No.: US 2005/0060780 P1
(43) Pub. Date: Mar. 17, 2005

(54) Cymbidium 'LOVELY VALLEY Peace in the World'

(76) Inventor: Takehiko Mukoyama, Yamanashi (JP)

Correspondence Address: MUKOYAMA ORCHIDS CO., LTD., 274 KUMANO, ENZAN, YAMANASHI, SUSUMU FURUYA, 404-0036, JP

(21) Appl. No.: 10/853,019

(22) Filed: May 25, 2004

(30) Foreign Application Priority Data
May 26, 2003 (JP) 15760

(51) Int. Cl. A01H 5/00

(52) U.S. Cl. PLT/263

(57) ABSTRACT

A distinct cultivar of Cymbidium plant named ‘LOVELY VALLEY Peace in the World’ characterized by upright growth habit with particular vigor, free flowering, bright yellow-green flower with red lip, that is not normally characteristic of Cymbidium. Plant size is medium and leaves are intermediate in diameter. The intensely colored yellow green flowers are carried on a fleshy, erect spike without any arching character. Flowers will maintain quality with no obvious senescence for a minimum of 60 days.

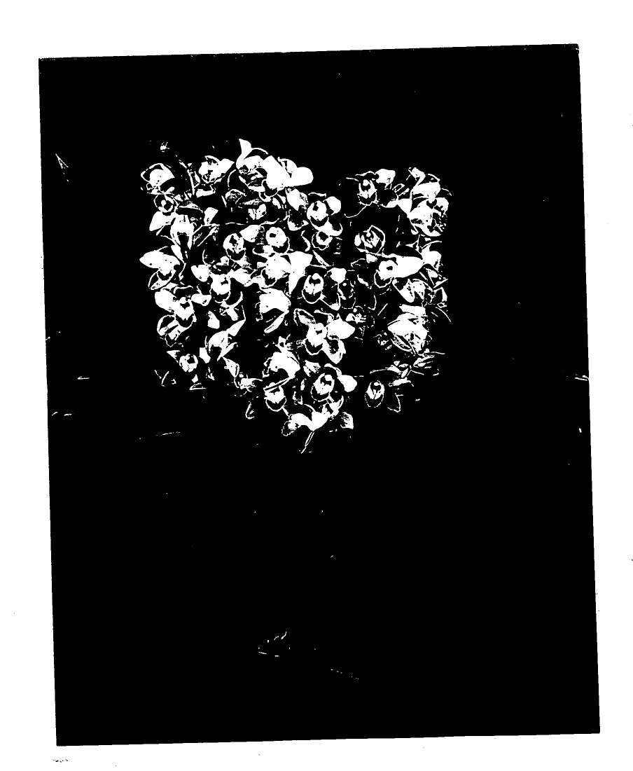

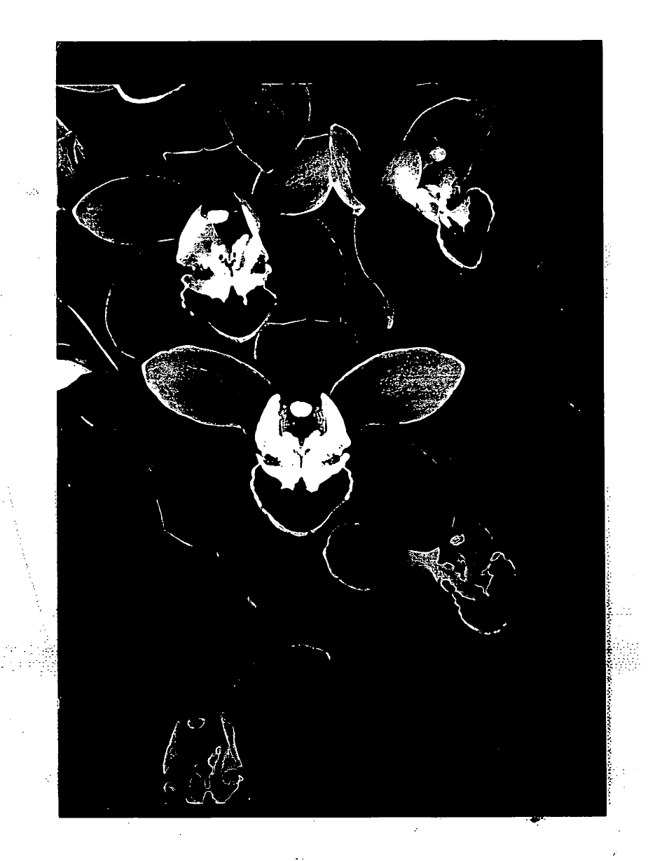

### BACKGROUND OF THE INVENTION

The present invention relates of a new and distinct cultivar of Cymbidium plant, botanically known as Lovely Valley (Cymbidium Dolores Hoyt ‘Lemon Light’×Cymbidium Valley Zenith), registered with The Royal Horticultural Society in England in 2001, hereinafter referred to by the cultivar name ‘LOVELY VALLEY Peace in the World’

The new cultivar is the product of a crossing using the above parents by the inventor in 274 Kumano, Enzan, Yamanashi 404-0036 Japan. Pollination was effected on Mar. 15th, 1995. The inventor's object was to produce a Cymbidium that has

(a) Vigorous and rapid growth habit

(b) Good spike habit

(c) Yellow green flowers with red lips

The cultivar ‘LOVELY VALLEY Peace in the World’ appeared in January 2000 as a single outstanding plant among a large seedling population of the grex at Mukoyama Orchids Co., Ltd., an orchid nursery owned by the applicant in 274 Kumano, Enzan, Yamanashi 404-0036 Japan.

Comparison testing by the inventor at Mukoyama Orchids Co., Ltd. has established the following differences between the Cymbidium ‘LOVELY VALLEY Peace in the World’ and other siblings from the same parentage:

(1) Plants of ‘LOVELY VALLEY Peace in the World’ grow faster under comparable conditions than any other seedling from the same crossing.

(2) The plant of ‘LOVELY VALLEY Peace in the World’ was among the first seedlings from the crossing to bloom with an initial inflorescence of flowers fully open on Jan. 28th, 2000 which is four years and ten months after pollination.

(3) Flowers of ‘LOVELY VALLEY Peace in the World’ with a horizontal natural spread of 7.8 cm and a vertical natural spread of 5.8 cm are larger in both dimensions than all other seedlings from the same crossing.

(4) The brightness yellow green sepals and petals of ‘LOVELY VALLEY Peace in the World’ are unlike all other seedlings in the crossing.

(5) Leaves of ‘LOVELY VALLEY Peace in the World’ are upright type and are in the range of 21-30 mm and 75-105 cm length when mature.

Asexual propagation by division and tissue culture has been carried out at laboratories of Mukoyama Orchids Co., Ltd. in Yamanashi, Japan. Plants of ‘LOVELY VALLEY Peace in the World’ are stable and have bloomed without any phenotypic variation being observed.

### SUMMARY OF THE INVENTION

After discovery in January 2000, ‘LOVELY VALLEY Peace in the World’ has been observed under a wide range of environmental conditions. No mutation or variance in the genotype has been observed as result of vegetative division or tissue culture.

The following traits have been observed on all bloomings since January 2000 and are considered unique to the cultivar ‘LOVELY VALLEY Peace in the World’. In combination they distinguish ‘LOVELY VALLEY Peace in the World’ as a new and distinct cultivar:

(I) Rapid growth rate

(II) Longer lasting flowers, at 60 days, uncut, than all other seedlings from the same parentage.

(III) Larger flowers, at 61-80 mm horizontal natural spread, than all other seedlings from the same parentage.

(IV) The plant produce 2 to 3 spikes per mature pseudobulb.

(V) The plant carries upright spikes of up to sixteen rounded, bright yellow-green flowers.

(VI) The plant carries normally five-six leaf pairs (10-12 leaves) on each newly mature pseudobulb. The bulbs and spike show slight traces of anthocyanin pigment.

### BRIEF DESCRIPTION OF THE PHOTOGRAPH

Accompanying color pictures illustrate the overall appearance of the new cultivar with colors as true as is possible to obtain in color reproductions of this type. The pictures were taken in January 2000.

### DETAILED BOTANICAL DESCRIPTION

In the following description color references are made to The Royal Horticultural Society Color Chart. Observation and measurements are made from plants grown at 274 Kumano, Enzan, Yamanashi 404-0036 Japan in plastic topped greenhouse with screen sidewalls. Night temperatures normally range between 10-20 degrees Celsius annually and day temperatures between 15-28 degrees Celsius.
(A) Botanical classification: Cymbidium Lovely Valley (Dolores Hoyt ‘Lemon Light’×Valley Zenith) ‘Peace in the World’.
(B) Parentage:
   Female parent.—Dolores Hoyt ‘Lemon Light’, a hybrid of Cymbidium King Arthur×Cymbidium Endre Ostbo.
(C) Parentage:
   Male parent.—Valley Zenith, a hybrid of Cymbidium Miretta×Zumma Boyd.
(D) Propagation: by vegetative division and tissue culture. Time interval from excision of apical and lateral meristern till delivery of 10,000 rooted plants in vitro. This seems very precise maybe: approximately 400 days.
(E) Plant description:
   Shape.—Upright leaves with strongly erect flower spikes arising from above ground pseudobulbs of approximately 60 mm height. Mature plants, multispiking in 18 cm diameter pots.
   Height.—Approximately 75-105 cm from the base of pseudobulb to tip of leaf.
   Vigor.—Extremely vigorous with resistance to transplant shock.
   Pseudobulbs.—Normally not more than six per 18 cm pot. Shape is slightly elongate, circular, about 31-60 mm in height. Dark green with low levels of anthocyanin.
Flower description:
   Arrangement.—Medium bright yellow green flowers arranged bilaterally on the raceme. Typically the flower count will average 11-20 when the plant is 36 months from in-vitro deflasking. Flowering season is mid-winter. Usually double-triple inflorescences will be produced contemporaneously on each newly matured pseudobulb and the flowers are persistent.
   Longevity.—Inflorescences typically last in excess of 60 days in a wide range of environmental conditions uncut.
   Size.—Horizontal natural spread 61-80 mm. Vertical natural spread 41-60 mm.
   Petals.—length 50-60 mm.
   Petals.—width 20-25 mm.
   Petals.—color RHS Color Chart 3305.
   Dorsal sepals.—length 40-50 mm.
   Dorsal sepals.—width 25-30 mm.
   Dorsal sepals.—color RHS Color Chart 150A.
   Lateral sepals.—length 50-60 mm.
   Lateral sepals.—width 20-25 mm.
   Lateral sepals.—color RHS Color Chart 150B.
   Labellum.—length 30-40 mm.
   Labellum.—width 20-25 mm.
   Labellum.—color RHS Color Chart 145D labellum, stitched in red (RHS Color Chart 60A). which coalesces at the margins.
   Gynandrium.—color RHS Color Chart 159B.
Fragrance: Slight.
   Spike.—Strong and erect without any arching. Length about 74 cm with a diameter of 11 mm. Light-green with slight trace of anthocyanin pigment. Membraneous sheaths persistent.
   Disease resistance.—No observations have been made.

1. It is claimed that is a new variety of pot plant Cymbidium named ‘LOVELY VALLEY Peace in the World’ as illustrated and described.
